(19) United States
(12) Patent Application Publication
(10) Pub. No.: US 2003/0194385 A1
(43) Pub. Date: Oct. 16, 2003

(54) PHARMACEUTICAL FORMULATIONS CONTAINING SOLUBILIZED HYDROQUINONE, SALICYLIC ACID AND HYDROCORTISONE FOR THE TREATMENT OF MELASMA AND RELATED DERMATOLOGICAL PROBLEMS

(75) Inventor: Mr. Murray Gruber, Roslyn, NY (US)

Correspondence Address: DARBY & DARBY P.C., P. O. BOX 5257, NEW YORK, NY, 10150-5257

(73) Assignee: Lila Gruber Research Foundation, Roslyn, NY (US)

(21) Appl. No.: 10/354,435

(22) Filed: Jan. 29, 2003

Related U.S. Application Data
(60) Provisional application No. 60/352,651, filed on Jan. 29, 2002.

(51) Int. Cl. A61K 7/42; A61K 31/60; A61K 31/05

(52) U.S. Cl. 424/59; 514/159; 514/731

(57) ABSTRACT

### Abstract of the Disclosure

There are provided dermatological compositions achieved by combining solubilized, stabilized hydroquinone salicylic acid and hydrocortisone by dissolving the three ingredents in dimethyl isosorbide. These preparations are intended for the treatment of melasma and related disorders of the skin such as freckles, senile lentigines, chloasma, ultraviolet induced dyschromia and discoloration resulting from ingestion of oral contraceptives or hormonal replacement therapy.

### Detailed Description of the Invention

### CROSS REFERENCE TO RELATED APPLICATIONS

This application claims priority under 35 U.S.C. § 119(e) from Provisional Application No. 60/352,651, filed January 29, 2002.

### FIELD OF THE INVENTION

The present invention relates generally to pharmaceutical and cosmetic products, more specifically to those preparations incorporating solubized, stabilized hydroquinone, salicylic acid and hydrocortisone which when applied topically produces reversible depigmentation of human skin.

### BACKGROUND OF THE INVENTION

Hyperpigmentation of the skin in humans can be the cause of much mental distress. Hypermelanotic areas, especially on the face, can result in a marked psychological and cosmetic disability.

The efficacy and safety of hydroquinone as a bleaching agent for the treatment of melasma and related skin disorders has long been known and detailed in the medical literature.

Hydroquinone (C ₆ H ₆ 0 ₂ ) is an oxidizing agent in the form of white, odorless, tasteless solid crystals. Hydroquinone inhibits the conversion of tyrosine to melanin. See C. Denton, et al., Inhibition of Melanin Formation by Chemical Agents , J. Investigative Dermatology, 18: 119-135 (1952); K. Jimbow, Mechanism of Depigmentation by Hydroquinone , J. of Investigative Dermatology, 62: 436-449 (1974). Thus it does not truly "bleach" pigment, but inhibits the synthesis of melanin.

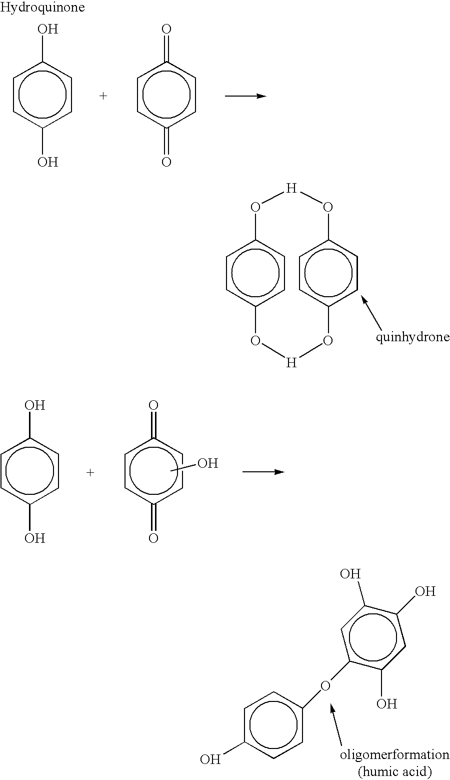

Hydroquinone has been formulated into creams, lotions and gels to produce effective skin lightening products. However, hydroquinone is known to be extremely unstable when incorporated in creams and lotions.

The major disadvantage of hydroquinone containing products has been the tendency of hydroquinone to oxidize and turn brown, resulting in the discoloration of the final product into which it is incorporated. Oxidization occurs when the pharmaceutical or cosmetic preparation is exposed to oxygen, metals, light and/or heat. When oxidation and browning occur, different molecules emerge and more complex reactions may result. Occasionally, black discoloration of the skin is reported. Besides these reactions of the skin, hydroquinone has been implicated as a cause of brown discoloration of the nails. This discoloration apparently was due to a deposition on the nail of brown products of hydroquinone oxidation. See Fitzpatrick TB, Arndt KA, El Mofty AM, Pathak MA: Hydroquinone and psoralens in the therapy of hypermelanosis and vitiligo. Arch Dermatol 93:589-600, 1966. Once the preparation is discolored, degraded forms appear and the safety, efficacy, purity, identity, quality, strength and integrity of the finished product is compromised. Attempts to suppress oxidation, decomposition discoloration of the manufactured product by avoiding exposure to oxygen during manufacture, and packaging in air impermeable squeeze tubes have not solved the problem of degradation.

Hydroquinone compositions are most effective when the active agent, hydroquinone, is released quickly. For such quick release, hydroquinone in this present invention is uniquely solubilized and not in particulate dispersion. In commercially available creams and lotions, the hydroquinone is in particulate dispersion. It is known in the art that hydroquinone is readily soluble in organic solvents such as ethyl alcohol, isopropyl alcohol, propylene glycol, and to a much lesser extent in ether, glycerol and is relatively insoluble in water. Some hydroquinone preparations contain high concentrations of alcohol, but have a tendency to be drying and irritating to the skin.

In an attempt to overcome the problem of oxidation, hydroquinone creams and lotions invariably contain a known allergen, sodium bisulfite, which acts as a reducing agent to inhibit oxidation. Sodium bisulfite may cause serious allergic type reactions (e.g. hives, itching, wheezing, anaphylaxis, severe asthma attack) in certain persons. a) See Package Inserts Lustra Medicis Scottsdale, Arizona, b) Elderquin and Solarquin ICN Costa Mesa, Calif.

Instability has posed a problem in formulating compositions containing hydroquinone, yet hydroquinone continues to be used as it is safe and effective. See The Merck Index (1989) 11 ^{th} ed. 762-3; Eastman Co., Hydroquinone Bulletin (1999). Hydroquinone is presently the only agent that is recognized by the US Food & Drug Administration as a safe and effective modality for lightening the pigmentation of the skin. Skin lighteners, including those containing hydroquinone, are regulated as drugs. Formulations containing more than 2.0% hydroquinone, but not more than 4.0 % are regulated as prescription drugs. See P. Engasser, and H. Maibach, Cosmetics and Dermatology: Bleaching Creams , Am. Acad. Dermatol. S: 143-47 (1981).

### SUMMARY OF THE INVENTION

There is a need in the art for solubilized, stabilized hydroquinone compositions in which the hydroquinone does not readily oxidize. The present invention provides a method of dissolving hydroquinone and stabilizing it in dimethyl isosorbide. The present invention also provides a topical dermatological composition comprising solubilized stabilized hydroquinone, salicylic acid and hydrocortisone in dimethyl isosorbide. In addition, the present invention completely eliminates the known disadvantages of hydroquinone therapy by stabilizing hydroquinone and without incorporation of sodium bisulfite and without the use of organic solvents such as ethyl alcohol.

The composition of the present invention is intended for the treatment of melasma and related disorders of the skin such as freckles, senile lentigines, chloasma, ultraviolet light induced dyschromia and discoloration resulting from ingestion of oral contraceptives or hormonal replacement therapy.

In one aspect the present invention provides a topical pharmaceutical formulation for treating a patient suffering from melasma and related dermatological conditions comprising an amount of solubilized, stabilized hydroquinone effective to treat said conditions.

In another aspect the present invention provides a topical pharmaceutical formulation for treating a patient suffering from melasma and related dermatological conditions comprising hydroquinone, salicylic acid and hydrocortisone, wherein the amounts of said hydroquinone, salicylic acid and hydrocortisone are solubilized and stabilized and in combination are effective to treat said conditions.

These and other aspects of the present invention will be apparent to those of ordinary skill in the art in light of the present description and claims.

### DETAILED DESCRIPTION OF THE INVENTION

All patent applications, patents and literature references cited herein are hereby incorporated by reference in their entirety.

The present invention utilizes dimethyl isosorbide to solubilize and stabilize hydroquinone without the addition of sodium bisulfite or the use of organic solvents. Dimethyl isosorbide is a unique solubilizer, emulsifier and emollient with unusual properties. It is practically non-toxic, water soluble, oil soluble, has a high boiling point and low freezing point, is miscible with many organic solvents, pH stable, non-greasy, non-drying, non-irritating, colorless, practically odorless, inert and non-volatile. The hydroquinone and dimethyl isosorbide are the major components and are therapeutically effective when employed by themselves with nothing added. Dimethyl isosorbide for use in the present invention can be obtained from numerous commercial sources such as Uniquema, New Castle, Delaware, Aldrich Chem. Co., Allentown, PA. Hydroquinone is used in the present invention at effective amounts between about 2% and about 10% and can be obtained from numerous commercial sources such as Eastman Chemical Co., Kingsport, TN 37662 and Rhodia Inc., Cranbury, NJ 08512.

A preferred formulation contains the following components. Hydroquinone, salicylic acid, hydrocortisone, sun screens and Vitamins C and E. When dissolved in dimethyl isosorbide, these components are "solubilized" and "stabilized". Salicylic acid is an aid in effecting penetration of hydroquinone. The hydrocortisone is an auxiliary medicament to overcome possible irritation and allergenicity. See Bentley-Phillips B, Bayles MAH: Cutaneous reactions to topical applications of hydroquinone. Results of a 6-year investigation. SA Med J 49: 1391-1395, 1975. The sunscreens are adjuncts to maintain the depigmentation obtained which is adversely affected by sunlight. See Vazquez M. Sanchez IL. The efficacy of a broad-spectrum sunscreen in the treatment of melasma. Cutis. 1983: 32:92-06. The vitamins C and E provide some antioxidant activity but primarily reduce erythemia and irritation.

The compositions disclosed herein may further comprise salicylic acid. The combination of salicylic acid with hydroquinone produces significantly more rapid depigmentation than achieved by hydroquinone used alone. Salicylic acid (C ₇ H ₆ 0 ₃ ) is a keratolytic agent in the form of white crystals, acrid in taste, odorless and stable in air. The outer layer of the skin is formed from dead cells composed largely of a substance known as keratin. Substances that promote the removal or sloughing off of excess keratin are known as keratolytic agents. Since salicylic acid acts by removing this horny layer it is classified as a keratolytic agent and a dermal penetrant. See D. Burrows, Therapeutics III - Quarterly Review , Br. J. Dermatol. 80: 550-53 (1968). Generally, enhanced penetration of the skin is expected for steroid preparations compounded with salicylic acid. See L. Krochmal, Topical Corticosteroid Compounding: Effects on Physicochemical Stability and Skin Penetration Rate , J. Am. Acad. Dermatol. 21: 979-84 (1989). Salicylic acid causes shedding of the keratin layer of the epidermis and facilitates penetration of the skin, with medicaments with which the salicylic acid is combined. When incorporated into a water miscible, water washable type of base, salicylic acid becomes readily and quickly available to the skin with the result that extremely low concentrations may be used. Salicylic acid can be prepared from wintergreen oil or synthesized by dissolving phenol in sodium hydroxide and heating 130°C. See U.S. Dispensatory (p1207) 25th Edition Olson & Ferrer. Salicylic acid for use in the present invention can also be purchased from numerous commercial sources such as EM Labs, Hawthorne, NY and Universal Preserv. A Chem, Edison, NJ.

Salicylic acid has incompatibilities characteristic of organic acids. Thus it causes emusified bases, such as vanishing creams to separate when the acid is incorporated. One embodiment of the present invention is a gel, not an emulsified cream or lotion. Therefore, it does not separate. Another embodiment is a cream, but it is a special formulation that likewise does not separate. The basis of the cream is hydrogenated vegetable oil NF and the absence of water.

The composition of this invention may further contain hydrocortisone. Hydrocortisone in the composition acts as an anti-inflammatory agent for which it is well known. Hydrocortisone for use in the present invention can be purchased from sources such as Upjohn, Kalamazoo, Michigan and Spectrum, Gardena, California.

The composition may also contain broad spectrum sunscreens to prevent repigmentation caused by the sun's UVB rays. See JA Parish, et al., UVA, Biological Effects of Ultraviolet Radiation with Emphasis on Human Responses to Longwave Ultraviolet , Plenum Press 151 (1978). Effective amounts of sunscreens for use in the present invention would range between about 6% and about 8% in dimethyl isosorbide.

COMMERCIAL SOURCES FOR SUNSCREENS:

a) EM Labs, Hawthorne, NY

b) Florasynth Co., Springfield, NJ

SUNSCREEN SOLUBILIZED (W/W IN DIMETHYL ISOSORBIDE:

1. Oxybenzone 6.0%

2. Octylsaliylate 6.0%

3. Octylmethoxycinnamate 8%

The amount of hydrocortisone may broadly range between about 1/4% and about 3% and preferably between about 1/2% and about 1%. The amount of salicylic acid for use in the composition may broadly range between about 1/2% and about 10% and preferably between about 1/2% and about 4%.

Hydrogenated vegetable oil is refined, bleached oil. Nationall Formulary XVII Pg. 1994 (available from Proctor & Gamble, Cincinnati, Ohio).

Dimethyl isosorbide may be broadly present in an amount ranging between about 10% and 95% and most preferably in an amount between about 70% and 78%. According to yet another embodiment, the solvent is 2-methyl, 1,3 propanediol. (Mpdiol Glycol). It is not as effective as dimethyl isosorbide, but provides an economic benefit. Mpdiol glycol is available from Lyon Dell, Newton Square, PA. Mpdiol concentration can range from 5% to 30% and can be used alone or in equal parts with dimethyl isosorbide.

Pursuant to the present invention, when hydroquinone is dissolved in dimethyl isosorbide it provides a uniform, stable preparation, which assures safety, quality, purity, identity, strength and integrity of the hydroquinone and avoids the common brown deterioration, discoloration and subsequent decomposition of components. Similarly, salicylic acid, when dissolved in the dimethyl isosorbide, provides a uniform, stable preparation that assures safety, quality, purity, identity, strength and integrity of the salicylic acid. Both the gel and cream, of the examples presented below, are non-reactive with the medicaments incorporated therein. The gel is thixotropic (shaking the container restores the thick gel to a more flowing medium), stable during storage and easily subjected to shear when spread. They show little or no viscosity change in the temperature range of 4-38°C, they are long lasting upon application, yet easy to remove.

In a further embodiment, the composition may contain various additives well known to one of ordinary skill in the art. Examples of additives include emulsifiers, waxes, oils, fatty acids, moisturizers, colorants, fragrances, vitamin A, Vitamin C and antioxidants. Listed below are non-limiting examples of additives for use in the present invention.

Additives:

Emulsifiers - including stearic acid, cetyl alcohol, stearyl alcohol, polysorbate 20.

Emollients - ceraphyl 230, olive oil, sesame oil, dimethicone, PEG75

Humectant - glycerin, propylene glycol

There are numerous other substances which could be incorporated dependent only upon the expertise of the formulator. In general, the addition of other substances should not exceed 5%. Suitable additives are listed in the CTFA Cosmetic Ingredient Handbook - 1998 Library of Congress Catalog #88-071506. Any additives must be carefully selected to avoid compromising the stability, safety, quality, purity, strength and integrity of the active ingredients. Many chemicals, emulsifiers, excipients, sunscreens, etc. could be added to enhance and improve the basic formula. In addition, the resultant products may be tested to insure stability and efficacy.

The final formulations may be packaged in containers impervious to sunlight for distribution to patients. To assure optimum stability, a nitrogen blanket may be provided during manufacture and the containers may also be protected with a nitrogen filled head space. Neither procedure, however, is absolutely necessary to obtain the required stability requirements of the therapeutic composition of the present invention.

For optimum contact with the epidermis and best therapeutic effect, the skin should be slightly moistened prior to application of the gel or cream.

Unlike the usual creams, salves and lotions, the compositions of this invention do not contain any petrolatum, propylene glycol, isopropyl palmitate or similar greasy substances. Dimethyl isosorbide provides an elegant, lubricating, non-greasy non-alcoholic, non-sticky, nonstaining emollient and skin-softening vehicle for these medications.

The compositions of this invention are compounded employing techniques well known in the pharmaceutical and cosmetic arts. The hydroquinone is dissolved in the dimethyl isosorbide at room temperature in a suitable stainless steel mixing vessel. The additional ingredients are subsequently added and similarly solubilized. This is a cold process, with no milling or heat required.

When used to treat the conditions described above, an effective amount of the formulation, which is a thin application as a light film to the affected areas is applied to the skin. The formulation may be applied 2 times a day and can be used for 90 days or until darkened skin returns to normal with maintenance applications thereafter.

The following examples are intended to illustrate more specifically the embodiments of the invention. It will be understood that while the invention as described therein is a specific embodiment, the description and the examples are intended to illustrate and not limit the scope of the invention. Other aspects, advantages and modifications within the scope of the invention will be apparent to those skilled in the art to which the invention pertains.

Example 1 - Gel Formulation

| | % by weight |
|---|---|
| Dimethyl Isosorbide | 78 |
| Hydroquinone | 4 |
| Salicylic Acid | 2 |
| Ceraphyl | 1 |
| Octylmethoxycinnamate | 7 |
| Octylsalicylate | 4 |
| Oxybenzone | 3 |
| Hydrocortisone | 1 |
| Total | 100 |

Example 2 - Cream Formulation

| | % by weight |
|---|---|
| Dimethyl Isosorbide | 20 |
| Hydroquinone | 4 |
| Salicylic Acid | 2 |
| Octylmethoxycinnamate | 7 |
| Octylsalicylate | 4 |
| Oxybenzone | 3 |
| Hydroxypropyl Cellulose | 0.5 |
| Bisabalol | 0.5 |
| Hydrogenated Vegetable Oil N.F. | 59 |
| Total | 100 |

Example 3 - Gel Formulation

| | % by weight |
|---|---|
| Dimethyl Isosorbide | 68 |
| Mpdiol glycol | 10 |
| Hydroquinone | 4 |
| Salicylic Acid | 2 |
| Ceraphyl | 1 |
| Octylmethoxycinnamate | 7 |
| Octylsalicylate | 4 |
| Oxybenzone | 3 |
| Hydrocortisone | 1 |
| Total | 100 |

Both the gel formulations of Examples 1 and 3 and the cream formulation of Example 2, were packaged in clear glass vials. Three vials of each formulation were exposed to direct sunlight for three months to determine if there was any separation of the preparations or decomposition as evidenced by "browning." There was no separation of the gels or cream and no color change of the hydroquinone content. Simultaneously, three other vials of hydroquinone and salicylic acid gels and cream formulations, without sodium bisulfite had their caps removed. They were subject to air exposure at ambient temperature, for three months to evaluate separation or decomposition. There was no "browning" or any color change whatsoever and no separation of the gels or cream. Additionally, three vials of the same gels and cream formulations were subjected alternately to elevated (40°C) and freezing temperature (4°C) for three months. No separation of the formulations or deterioration as evidenced by "browning" or other color change was detected.

The purpose of these experiments was to determine if there was any decomposition of the hydroquinone by direct air, sunlight and heat evidenced by "browning." Additionally, the tests demonstrated that sodium metabisulfite, usually present in such formulations, was not essential or critical as a stability enhancer. Accordingly, compositions prepared in accordance with this invention possess a stability that far exceeds the normal requirements for pharmaceutical products based upon their normally expected commercial life even if improperly stored.

### REFERENCES

Denton CR, Lerner AB, Fitzpatrick TB: Inhibitin of melanin formation by chemical agents. J Invest Dermatol 18:119-135, 1952.

Arndt KA, Fitzpatrick TB: Topical use of hydroquinone as a depigmenting agent. JAMA 194:965-967, 1965.

Spencer MC: Topical use of hydroquinone for depigmentation. JAMA 194:962-964, 1965.

Arndt KA, Fitzpatrick TB. Topical use of hydroquinone as a depigmenting agent. JAMA, 1965:194:965-967.

Fitzpatrick TB, Arndt KA, el Motty AM. Pathak MA. Hydroquinone and psoralens in the therapy of hypermelanosis and vitiligo. Arch Dermatol. 1966: 93:589-600.

Jimbow K, Obata H, Pathak MA, Fitzpatrick TB, Mechanism of depigmentation by hydroquinone, J Invest Dermatol 62:436-449, 1974.

Kligman AM, Willis I: A new formula for depigmenting human skin. Arch Dermatol 111:40-48, 1975.

Engasser PE. Malbach HI. Cosmetics and dermatology: bleaching creams. J Am Acad Dermatol. 1981:5:143-147.

Gano SE, Garcia RL. Topical tretinon, hydroquinone and betamethasone valerate in the therapy of melasma. Cutis. 1979:23:239-241.

Sanchez IL. Vazquez M.A. hydroquinone solution in the treatment of melasma. Int J. Dermatol. 1982:21:55-58.

Glenn M. Grimes PE, Pitt E. Chalet M., Kelley AP. Evaluation of clinical and light microscopic effects of various concentrations of hydroquinone. Clin Res 1991:39:83A. Abstract.

What is Claimed is:

1. A topical pharmaceutical formulation for treating a patient suffering from melasma and related dermatological conditions comprising an amount of solubilized, stabilized hydroquinone effective to treat said conditions.

2. The pharmaceutical formulation of claim 1 further comprising an amount of solubilized, stabilized salicylic acid effective to treat said conditions.

3. The pharmaceutical formulation of claim 2 further comprising an amount of solubilized, stabilized hydrocortisone effective to treat said conditions.

4. The pharmaceutical formulation of claim 3 wherein said formulation is solubilized and stabilized by dimethyl isosorbide.

5. The pharmaceutical formulation of claim 4 wherein said formulation is solubilized and stabilized by 2-methyl, 1, 3 propanedione.

6. The pharmaceutical formulation of claim 4 wherein said formulation is solubilized and stabilized by a mixture of dimehyl isosorbide and 2-methyl, 1, 3 propanedione.

7. The pharmaceutical formulation of claim 4 further comprising an effective amount of a sunscreen.

8. The pharmaceutical formulation of claim 7 wherein said formulation is in a form selected from a cream and a gel.

9. The pharmaceutical formulation of claim 8 further comprising an amount of a sunscreen effective to treat said conditions.

10. A pharmaceutical formulation for treating a patient suffering from melasma and related dermatological conditions comprising hydroquinone, salicylic acid and hydrocortisone, wherein the hydroquinone, salicylic acid and hydrocortisone are solubilized and stabilized and in combination are effective to treat said conditions.

11. The pharmaceutical formulation of claim 10 further comprising an amount of a sunscreen effective to treat said conditions.

12. A method for treating a patient suffering from melasma and related dermatological conditions comprising topically administering to said patient in need of such treatment the pharmaceutical formulation of claim 9.

13. A method for treating a patient suffering from melasma and related dermatological conditions comprising topically administering to said patient in need of such treatment the pharmaceutical formulation of claim 1.
